(19) United States
(12) Patent Application Publication
(10) Pub. No.: US 2009/0137890 A1
(43) Pub. Date: May 28, 2009

(54) DEVICES TO MONITOR GLUCOSE LEVELS AND ISCHEMIA

(76) Inventors: JOHN E. BURNES, Coon Rapids, MN (US); EDUARDO N. WARMAN, Maple Grove, MN (US)

Correspondence Address: MEDTRONIC, INC., 710 MEDTRONIC PARKWAY NE, MINNEAPOLIS, MN, 55432-9924, US

(21) Appl. No.: 11/945,875

(22) Filed: Nov. 27, 2007

(51) Int. Cl. A61B 5/00; A61B 5/02

(52) U.S. Cl. 600/365; 600/301; 600/481

(57) ABSTRACT

The disclosure relates to systems, methods, and devices for monitoring a patient's blood and cardiac condition. Patients with diabetes oftentimes wear diabetes management equipment (e.g., a glucose monitor, an external insulin pump, or a device having dual functionality). Such patients risk silent myocardial infarction. Herein described is regular cardiac ischemia/infarction monitoring—which if not monitored can lead to (silent) myocardial infarction. Moreover herein described are combined blood monitoring functionality and cardiac condition monitoring functionality via a single device, meaning that the patient is not required to wear additional equipment. Adding this functionality to already-existing equipment is significantly less invasive than requiring a patient to wear one piece of equipment to monitor his/her blood and a second piece of equipment to monitor his/her cardiac condition. This reduction in invasiveness can lead to significantly greater patient participation and compliance, which can improve health and save the lives of many patients.

### BACKGROUND

The present invention relates generally to diabetes management and ischemia monitoring.

Diabetes is becoming more and more prevalent in the United States and elsewhere in the world. Persons afflicted with diabetes are at increased risk for stroke, ischemic heart disease, peripheral vascular disease, neuropathy, and other dangerous conditions.

Moreover, persons afflicted with diabetes, metabolic syndrome, or insulin resistance may have neural damage, making them especially susceptible to silent myocardial infarction. Without conventional symptoms (e.g., chest pain, neck or jaw pain, arm pain, clammy skin, shortness of breath, nausea and vomiting, etc.), a person is much less likely to detect cardiac ischemia in its early stages. As a result, treatment is often delayed, leading to greater adverse health consequences.

### BRIEF DESCRIPTION OF THE FIGURES

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a perspective view of a system for managing a patient's diabetes and monitoring for cardiac ischemia/infarction in the patient.

FIG. 2 is a block diagram of an illustrative chemical glucose monitoring assembly.

FIG. 3 is an illustrative display that can be implemented in connection with a chemical glucose monitoring assembly such as that of FIG. 2.

FIG. 4 is a block diagram of an illustrative external insulin pump assembly.

FIG. 5 is a block diagram of an illustrative system for monitoring for cardiac ischemia/infarction.

FIG. 6 is a block diagram of an illustrative system adapted to chemically monitor glucose levels in a patient, administer insulin to the patient from an external reservoir, and monitor for cardiac ischemia/infarction in the patient.

### DETAILED DESCRIPTION OF ILLUSTRATIVE EMBODIMENTS

The following detailed description is exemplary in nature and is not intended to limit the scope, applicability, or configuration of the invention in any way. Rather, the following description provides practical illustrations for implementing exemplary embodiments of the present invention. Constructions, materials, dimensions, and manufacturing processes suitable for making embodiments of the present are known to those of skill in the field of the invention. Those skilled in the art will recognize that many of the examples provided have suitable alternatives that can be utilized.

Embodiments of the present invention provide systems, methods, and devices for managing a patient's diabetes and monitoring for cardiac ischemia/infarction in the patient. Patients with diabetes are often required to wear diabetes management equipment, such as a continuous chemical glucose monitor, an external insulin pump, or a device having the functionality of both. Such patients are often at risk for silent myocardial infarction. Embodiments of the present invention monitor for cardiac ischemia/infarction on a regular basis, which, if left un-checked, can lead to silent myocardial infarction. Moreover, embodiments of the present invention combine the diabetes management functionality with the cardiac ischemia/infarction monitoring functionality into a single device, meaning that the patient is not required to wear additional equipment. Adding this functionality to already-existing equipment is significantly less invasive than requiring a patient to wear one piece of equipment to manage his/her diabetes and a second piece of equipment to monitor for cardiac ischemia/infarction. This reduction in invasiveness can lead to significantly greater participation and compliance, which can improve the health of, and even save the lives of, many patients.

Many embodiments of the present invention provide additional benefits. For example, when some embodiments detect the presence of cardiac ischemia/infarction, they provide therapy (e.g., potassium, insulin, glucose, thrombolysis, etc.) to the patient. Another example is that when some embodiments detect the presence of cardiac ischemia/infarction, they alert the patient and/or a caregiver (e.g., via telemetry) of the abnormal or otherwise noteworthy situation.

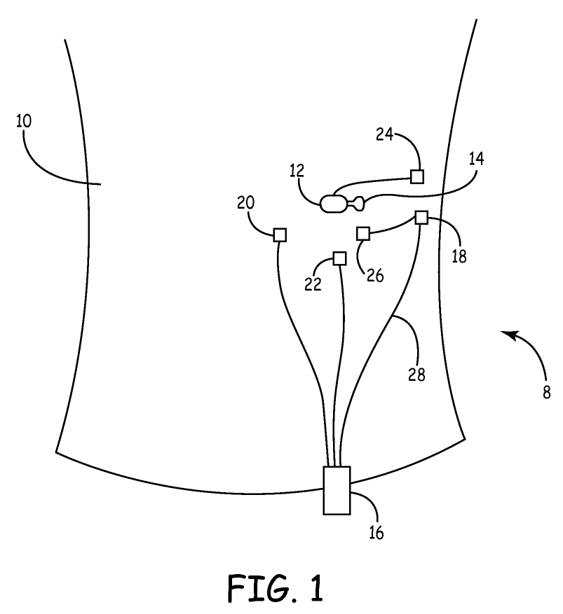

Some embodiments of the invention include the features shown in FIG. 1. FIG. 1 shows an illustrative system 8 for managing a patient's diabetes and monitoring for cardiac ischemia/infarction in the patient. The system 8 is shown on a patient 10. As shown, the system 8 includes a diabetes management device, a plurality of cardiac sensors, and cardiac monitoring circuitry. The diabetes management device of FIG. 1 is external to the patient. In some embodiments, diabetes management device is implantable.

The diabetes management device of FIG. 1 is adapted to chemically monitor glucose levels in the patient 10. The diabetes management device includes a chemical glucose monitoring assembly, which includes a glucose sensor unit 14, a transmitter 12, and a monitor/pump 16. Chemical glucose monitoring is the most reliable method of glucose monitoring. The glucose sensor unit 14 (which can comprise multiple sensors) can be inserted into the sub-cutaneous space or venous system of the patient 10. The glucose sensor unit 14 senses the blood glucose level in the patient 10 and generates electrical signals based on to the glucose concentration. The glucose sensor unit 14 can then provide some or all of those electrical signals to the transmitter 12, which can transmit some or all of the electrical signals it receives to the monitor/pump 16. In many embodiments, the transmitter 12 provides electrical signals to the monitor/pump 16 wirelessly. Glucose monitoring circuitry housed by the monitor/pump 16 can then interpret those electrical signals based on a previously determined calibration to determine a blood glucose value.

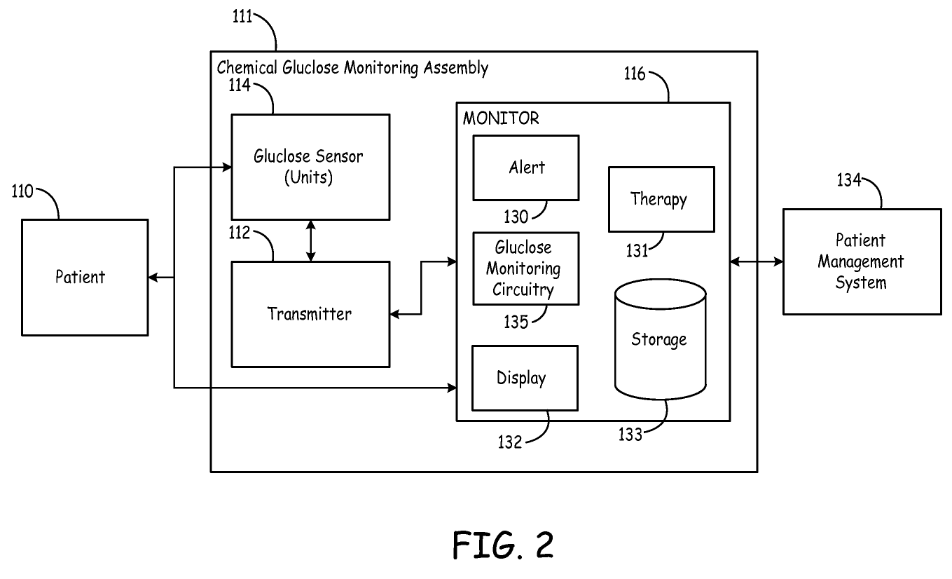

FIG. 2 is a block diagram of an illustrative chemical glucose monitoring assembly 111. Such an assembly 111 can perform a variety of functions with blood glucose values. For example, the monitor 116 of the assembly 111 can store information in storage 133 based on blood glucose values. Glucose monitoring circuitry 135 housed by the monitor 116 of the assembly 111 can compare blood glucose values with values from storage 133 to determine rates of change. The monitor 116 can include a display 132, which can display information based on blood glucose values. The monitor 116 can include a therapy-providing module 131, which can provide therapy (e.g., potassium, insulin, glucose, thrombolysis, etc.) to the patient 110 based on blood glucose values. The monitor 116 can include an alerting module 130, which can alert the patient 110 and/or a caregiver (e.g., via telemetry) of an abnormal or otherwise noteworthy situation based on blood glucose values. The monitor 116 can provide information to a patient management system 134 based on blood glucose values. Some monitors can retrieve information from patient management systems. Some chemical glucose monitoring assemblies perform one or more of the same functions as the assembly 111 of FIG. 2. Some chemical glucose monitoring assemblies can perform a variety of additional functions, depending on the patient's condition, the size and configuration of the assembly, the frequency of information gathering, and so on.

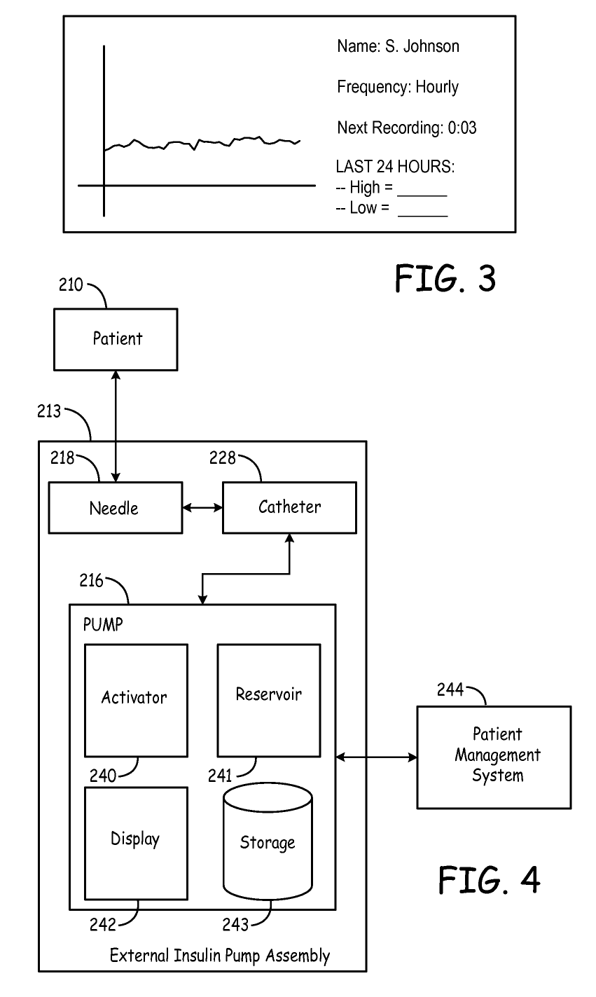

In many embodiments, chemical glucose monitoring assemblies function on a “continuous” basis. Blood glucose values can be provided to the monitor 116, e.g., once per minute can store and display, e.g., the patient's name, the number of collected blood glucose values during a given time (e.g., that day). In many continuous chemical glucose monitoring assemblies, the display 132 can display information as a graphic display that indicates the last several hours of recorded values, thereby showing any trends in the information over such time period. An illustrative display is provided in FIG. 3.

In many embodiments, the chemical glucose monitoring assembly 111 is programmable. In some such embodiments, a program can be created by a physician at his/her workstation and then synchronized with the monitor 116. That program can then be stored in storage 133. Some embodiments of the chemical glucose monitoring assembly 111 can be programmed via display 132. That program can be stored in storage 133.

Referring again to FIG. 1, the diabetes management device of FIG. 1 is also adapted to administer insulin to the patient 10 from an external reservoir. The diabetes management device includes an external insulin pump assembly, which includes the monitor/pump 16, a catheter 28, and a needle 18. The monitor/pump 16 can access an insulin reservoir that is external to the patient 10. In many embodiments, the external reservoir is housed by the monitor/pump 16.

FIG. 4 is a block diagram of an illustrative external insulin pump assembly 213. The pump 216 of the assembly 213 can be activated via an activator 240. In some embodiments, the activator 240 can be activated manually (e.g., after the patient 10 takes a glucose measurement). In some embodiments, the activator 240 can be activated automatically (e.g., according to a predetermined schedule, in response to a signal from a glucose monitor based on glucose level information, etc.). In some embodiments, the activator 240 of the external insulin pump assembly's pump 216 can be capable of being activated in one or more of the aforementioned ways, or in any suitable way.

Upon activation, the external insulin pump assembly 213 can administer insulin to the patient 210. The pump 216 can draw a desired and/or pre-programmed amount of insulin from a reservoir 241. In FIG. 4, the reservoir 241 is housed by the pump 216. When the pump 216 has drawn the desired and/or pre-programmed amount of insulin from the reservoir 241, the pump 216 can pump that insulin through the catheter 228 and into the patient 210 through the needle 218.

In many embodiments, the external insulin pump assembly 213 is programmable. In some such embodiments, a program can be created by a physician at his/her workstation and then synchronized with the pump 216. That program can then be stored in storage 243. Some embodiments of the external insulin pump assembly 213 can be programmed via a display 242. That program can be stored in storage 243. The display 242 of the pump 216 can display information related to administering insulin to the patient 210. In some embodiments, the pump 216 of the external insulin pump assembly 213 can communicate with a patient management assembly 244.

Though the diabetes management device of FIG. 1 may include both a glucose monitoring assembly and an external insulin pump assembly, many embodiments include only one or the other. Diabetes management devices can be used to (a) chemically monitor glucose levels in the patient, (b) administer insulin to the patient from an external reservoir, or (c) both. Some embodiments are adapted to only chemically monitor a patient's glucose levels. For example, a patient may employ a continuous chemical glucose monitor to obtain blood glucose values and then introduce insulin into his/her body via conventional syringe injection based on those blood glucose values. Some embodiments are adapted only to administer insulin to a patient from an external reservoir. For example, a patient may obtain blood glucose values via conventional pin-prick methods and then activate the external insulin pump assembly to introduce insulin into his/her body.

Referring again to FIG. 1, the system shown can also monitor for cardiac ischemia/infarction in the patient 10. The system can include one or more cardiac sensor units. A cardiac sensor unit is a collection of components, including one or more cardiac sensors, that contribute to collecting information concerning a patient's cardiac activity. Cardiac sensor units can provide some or all of the collected information (e.g., electrically) to cardiac monitoring circuitry housed by the monitor/pump 16. The cardiac monitoring circuitry is discussed in greater detail below. As shown, FIG. 1 includes cardiac sensors 20, 22, 24, 26. The cardiac sensors 20, 22, 24, 26 of FIG. 1 can be adapted to form one or more cardiac sensor units, which can collect information concerning cardiac activity of the patient 10. In some embodiments, a cardiac sensor unit can sense electrical activation of the heart in the form of an electrocardiogram (ECG) in order to monitor cardiac activity. The cardiac sensor unit generates electrical signals based on the myocardial potential differences and provides those electrical signals to cardiac monitoring circuitry housed by the monitor/pump 16. In some embodiments, cardiac sensors 20, 22, 24, 26 can include electrodes, one or more acoustic sensors, and/or one or more chemical sensors. Many embodiments include only one cardiac sensor unit.

A system for monitoring for cardiac ischemia/infarction can incorporate a greater or lesser number of cardiac sensors than A system according to FIG. 1. For example, a cardiac sensor unit can include cardiac sensors positioned on one or more of the monitor/pump 16, the glucose sensor unit 14, the transmitter 12, and/or the needle 18. A cardiac sensor unit with two or more of cardiac sensors positioned in any of the locations discussed herein can sense myocardial potential differences between those two or more locations. Cardiac sensor units with more than two cardiac sensors can often more accurately sense myocardial potential differences by, e.g., reducing the effect of noise or signal loss. In many instances, cardiac sensor units having one or more cardiac sensors positioned on the anterior of the patient 10 at heart level and one or more cardiac sensors positioned on the side or posterior of the patient 10 at heart level can sense myocardial potential differences most accurately. Especially advantageous cardiac sensor units include cardiac sensors positioned only on diabetes management equipment (e.g., two of the monitor/pump 16, the glucose sensor unit 14, the transmitter 12, and the needle 18). Systems with such cardiac sensor units minimize the amount of equipment patients are required to wear.

The cardiac sensor unit(s) can communicate information concerning the patient's cardiac activity to the cardiac monitoring circuitry in a variety of ways. Cardiac sensors 20, 22 are in electrical communication with the cardiac monitoring circuitry via conductors, so a cardiac sensor unit comprising those two cardiac sensors 20, 22 can communicate information to the cardiac monitoring circuitry via those conductors. Cardiac sensor 26 is in electrical communication with the cardiac monitoring circuitry by way of the needle 18 and catheter 28 of the external insulin pump assembly. A conductor couples cardiac sensor 26 to the external insulin pump assembly's needle 18 (which is made of conductive material), and from there, a conductor is coupled to the cardiac monitoring circuitry. Thus, a cardiac sensor unit comprising cardiac sensor 26 can communicate information to the cardiac monitoring circuitry via that path. Cardiac sensor 24 is in electrical communication with the cardiac monitoring circuitry by way to the chemical glucose monitoring assembly's transmitter 12. A conductor couples cardiac sensor 24 to the transmitter 12, and from there, the transmitter 12 is in wireless communication with the cardiac monitoring circuitry. Thus, a cardiac sensor unit comprising cardiac sensor 24 can communicate information to the cardiac monitoring circuitry via that path. Cardiac sensor units that employ one or more cardiac sensors can communicate information to the cardiac monitoring circuitry in one or more of the ways discussed herein or in any other suitable way.

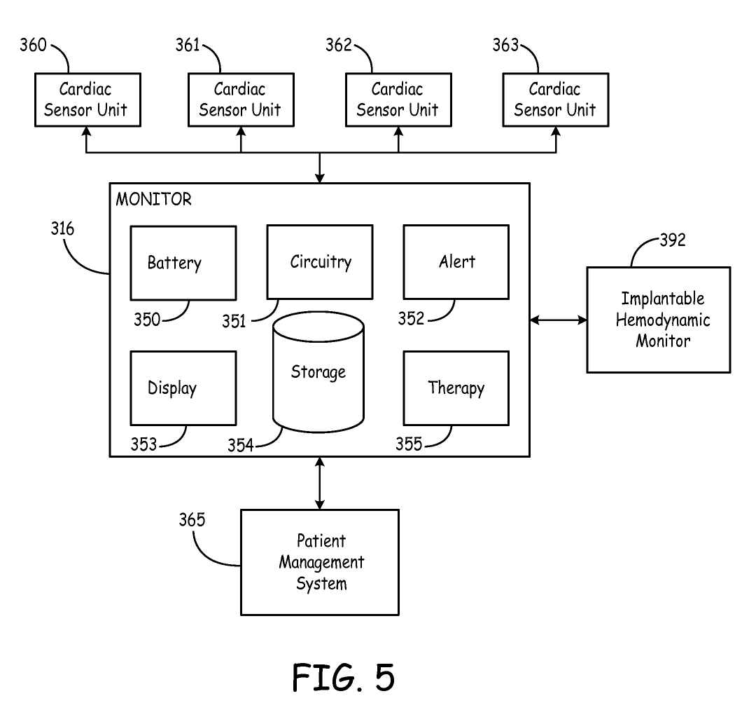

FIG. 5 is a block diagram of an illustrative system for monitoring for cardiac ischemia/infarction. The system includes four cardiac sensor units 360-363, which can employ any number and/or kind of the cardiac sensors discussed herein and which can be positioned in any of the locations discussed herein. As mentioned above, a greater or lesser number of cardiac sensor units can be used. The system also includes a monitor 316, which can have characteristics similar to those of the glucose monitors discussed herein. A system according to FIG. 5 includes an implantable hemodynamic monitor 392. In some embodiments, one or more cardiac sensors can be positioned on the implantable hemodynamic monitor 392. In some embodiments, the monitor 316 can communicate (e.g., two-way) with the implantable hemodynamic monitor 392. Such a system can be used for consolidating data retrieved by both devices, providing for enhanced sensing of cardiac ischemia/infarction and providing a single point of communication for interrogation (e.g., in a caregiver's office or by a home monitor). In some systems in which the monitor 316 can communicate with the implantable hemodynamic monitor 392, the implantable hemodynamic monitor 392 can identify the presence of ischemia/infarction and trigger a therapy response (e.g., potassium, insulin, glucose, thrombolysis, etc.) from one or more of the external components.

The monitor 316 can house a variety of components. The monitor 316 of FIG. 5 houses cardiac monitoring circuitry 351. The cardiac monitoring circuitry 351 may include all of the electronics for monitoring for cardiac ischemia/infarction. The monitor 316 may also include a battery 350 for powering the cardiac monitoring circuitry 351 and storage 354 accessible by the cardiac monitoring circuitry 351.

The cardiac monitoring circuitry 351 receives information from the cardiac sensor units 360-363 and can then assess whether that information indicates the presence of cardiac ischemia/infarction. If the cardiac monitoring circuitry 351 detects the presence of cardiac ischemia/infarction, it can trigger an alerting module 352 housed by the monitor 316. The alerting module 352 can alert the patient and/or a caregiver. The patient can then be examined to verify whether cardiac ischemia/infarction is present and, if necessary, begin receiving treatment. In some embodiments, if the cardiac monitoring circuitry 351 detects the presence of cardiac ischemia/infarction, it can trigger a therapy providing module 355 housed by the monitor 316. The therapy proving module 355 can provide therapy (e.g., potassium, insulin, glucose, thrombolysis, etc.) to the patient. In some embodiments, the cardiac monitoring circuitry 351 is adapted to provide at least some of the information it receives from the cardiac sensor units 360-363 to a patient management system 365. In some embodiments, the monitor 316 can retrieve information from the patient management system 365. In the embodiment of FIG. 5, the monitor 316 houses a display 353, which can display information based on the assessment of the cardiac monitoring circuitry 351. For example, the display 353 can display contact information for a caregiver in the event of detection of cardiac ischemia/infarction.

In many embodiments, some or all of the information provided by the cardiac sensor units 360-363 over a given duration (e.g., 24 hours) can be stored in storage 354. Like a Holter Monitor or Medtronic's Cardiac Compass®, this functionality allows caregivers to monitor and log parameters from an ECG such as heart rate, heart rate variability, and ischemic burden over various time intervals (e.g., day, night, 24 hours, during activity, during exercise).

In many embodiments, the cardiac monitoring system is programmable. In some such embodiments, a program can be created by a physician at his/her workstation and then synchronized with the monitor 316. That program can then be stored in storage 354. Some embodiments of the cardiac monitoring system can be programmed via display 353. That program can be stored in storage 354.

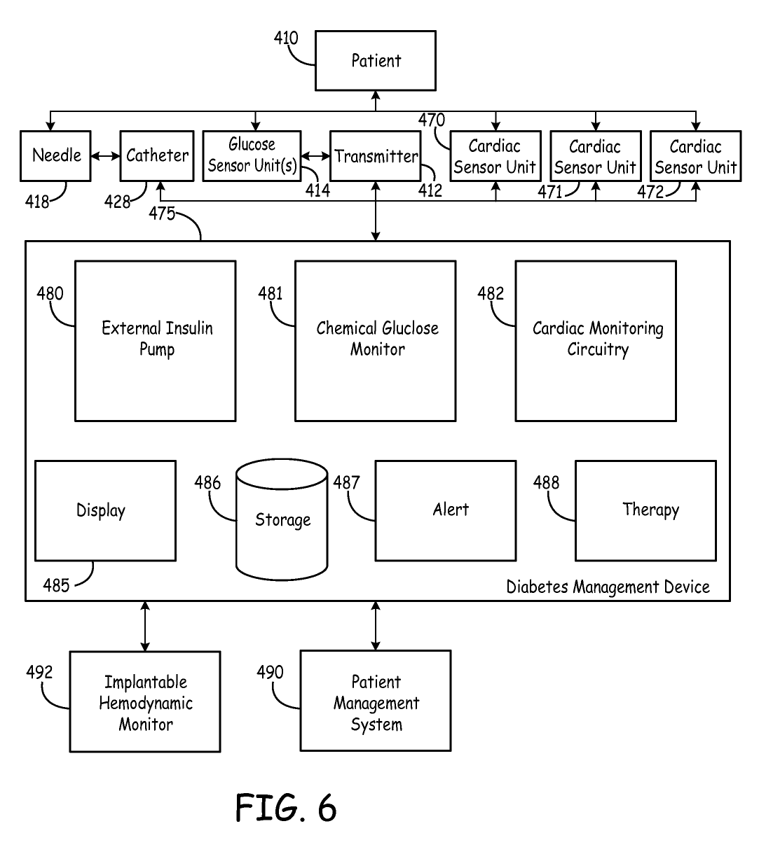

FIG. 6 is a block diagram of an illustrative system adapted to chemically monitor glucose levels in the patient 410, administer insulin to the patient 410 from an external reservoir, and monitor for cardiac ischemia/infarction in the patient 410. The system includes a diabetes management device 475, which includes, among other components, an external insulin pump 480, a chemical glucose monitor 481, and cardiac monitoring circuitry 482. The external insulin pump 480 can cooperate with a catheter 428, and a needle 418 to administer insulin to the patient 410 from a reservoir external to the patient 410, as discussed elsewhere herein. The chemical glucose monitor 481 can cooperate with a transmitter 412 and one or more glucose sensor units 414 to chemically monitor glucose levels in the patient 410 as discussed elsewhere herein. The cardiac monitoring circuitry 482 can cooperate with cardiac sensor units 470-472 to monitor for cardiac ischemia/infarction in the patient 410. The diabetes management device 475 of FIG. 6 further includes a display 485, storage 486, an alerting module 487, and a therapy providing module 488, the functions of which are discussed elsewhere herein. Some embodiments of the diabetes management device 475 can communicate with a patient management system 490 and/or an implantable hemodynamic monitor 492. Some diabetes management devices can perform a variety of additional functions, depending on the patient's condition, the patient's comfort level, the size and the configuration of the device, and so on.

Thus, embodiments of the present invention are disclosed. One skilled in the art will appreciate that the present invention can be practiced with embodiments other than those disclosed. The disclosed embodiments are presented for purposes of illustration and not limitation, and the present invention is limited only by the claims that follow.

1-17. (canceled)

18. A system for managing a patient's diabetes and monitoring for cardiac ischemia/infarction in the patient. comprising:
diabetes management means for chemically monitoring glucose levels in the patient and/or administering insulin to the patient from an external reservoir;
cardiac sensing means for collecting information concerning cardiac ischemia/infarction in the patient;
cardiac monitoring circuitry housed by the diabetes management means and adapted to receive at least some of the information concerning the cardiac activity from the cardiac sensing means; and
a therapy providing module for providing therapy to the patient, the therapy providing module being housed by the diabetes management means, wherein the cardiac monitoring circuitry is adapted to trigger the therapy providing module based on the information it receives concerning the patient's cardiac ischemia/infarction activity.

19. A system for managing a patient's diabetes and monitoring for cardiac ischemia/infarction in the patient, comprising:
diabetes management means for chemically monitoring glucose levels in the patient and/or administering insulin to the patient from an external reservoir;
cardiac sensing means for collecting information concerning cardiac ischemia/infarction in the patient;
cardiac monitoring circuitry housed by the diabetes management means and adapted to receive at least some of the information concerning the cardiac activity from the cardiac sensing means; and
an alerting module for alerting the patient and/or a caregiver, the alerting module being housed by the diabetes management means, wherein the cardiac monitoring circuitry is adapted to trigger the alerting module based on the information it receives concerning the patient's cardiac activity.

20. The system of claim 18, wherein the cardiac monitoring circuitry is adapted to receive information from the cardiac sensing means wirelessly.

21. The system of claim 19, wherein the cardiac monitoring circuitry is adapted to receive information from the cardiac sensing means wirelessly.

22. The system of claim 18, wherein the cardiac sensor unit comprises at least one cardiac sensor, adapted for positioning on and/or positioned on one of the following:
(a) a chemical continuous glucose monitoring assembly's glucose sensor unit,
(b) a chemical continuous glucose monitoring assembly's transmitter,
(c) a chemical continuous glucose monitoring assembly's monitor,
(d) an insulin pump assembly's pump,
(e) an insulin pump assembly's catheter,
(f) an insulin pump assembly's needle,
(g) an implantable hemodynamic monitor, or
(h) the patient's skin.

23. The system of claim 18, wherein the cardiac sensor unit comprises at least two cardiac sensors, each adapted for positioning on and/or positioned on one of (a)-(h).

24. The system of claim 18, further comprising means for providing at least some of the information provided to the cardiac monitoring circuitry to a patient management system.

25. The system of claim 18, further comprising means for providing at least some of the information provided to the cardiac monitoring circuitry to a display housed by the diabetes management device.

26. The system of claim 18, further comprising means for alerting the patient and/or a caregiver based on the information provided to the cardiac monitoring circuitry.

27. The system of claim 19, wherein the cardiac sensor unit comprises at least one cardiac sensor means adapted for positioning on and/or positioned on one of the following:
(a) a chemical continuous glucose monitoring assembly's glucose sensor unit,
(b) a chemical continuous glucose monitoring assembly's transmitter,
(c) a chemical continuous glucose monitoring assembly's monitor,
(d) an insulin pump assembly's pump,
(e) an insulin pump assembly's catheter,
(f) an insulin pump assembly's needle,
(g) an implantable hemodynamic monitor, or
(h) the patient's skin.

28. The system of claim 19, wherein the cardiac sensor unit comprises at least two cardiac sensors, each adapted for positioning and/or positioned on one of (a)-(h).

29. The system of claim 19, further comprising means for providing at least some of the information provided to the cardiac monitoring circuitry to a patient management system.

30. The system of claim 19, further comprising means for providing at least some of the information provided to the cardiac monitoring circuitry to a display housed by the diabetes management device.

31. The system of claim 19, further comprising means for providing therapy to the patient based on the information provided to the cardiac monitoring circuitry.

32. The system of claim 18, wherein the diabetes management device comprises an external insulin pump assembly having a pump, a catheter, and a needle, and wherein the at least one cardiac sensor is positioned on the pump, the catheter, and/or the needle.

33. The system of claim 18, wherein the diabetes management device comprises a chemical continuous glucose monitoring assembly having a glucose sensor unit, a transmitter, and a monitor, and wherein the at least one cardiac sensor is positioned on the glucose sensor unit, the transmitter, and/or the monitor.

34. The system of claim 18, wherein the diabetes management device comprises:
a chemical continuous glucose monitoring assembly having a glucose sensor unit, a transmitter, and a monitor and
an insulin pump assembly having a pump, a catheter, and a needle,
wherein the cardiac sensor unit comprises at least two cardiac sensors, each positioned on one of the glucose sensor unit, the transmitter, the monitor, the pump, the catheter, and the needle.

35. The system of claim 18, wherein the at least one cardiac sensor is positioned on an implantable hemodynamic monitor.

36. The system of claim 19, wherein the diabetes management device comprises an external insulin pump assembly having a pump, a catheter, and a needle, and wherein the at least one cardiac sensor is positioned on the pump, the catheter, and/or the needle.

37. The system of claim 19, wherein the diabetes management device comprises a chemical continuous glucose monitoring assembly having a glucose sensor unit, a transmitter, and a monitor, and wherein the at least one cardiac sensor is positioned on the glucose sensor unit, the transmitter, and/or the monitor.

38. The system of claim 19, wherein the diabetes management device comprises:
a chemical continuous glucose monitoring assembly having a glucose sensor unit, a transmitter, and a monitor and
an insulin pump assembly having a pump, a catheter, and a needle,
wherein the cardiac sensor unit comprises at least two cardiac sensors, each positioned on one of the glucose sensor unit, the transmitter, the monitor, the pump, the catheter, and the needle.

39. The system of claim 19, wherein the at least one cardiac sensor is positioned on an implantable hemodynamic monitor.
